(19) United States
(12) Patent Application Publication
(10) Pub. No.: US 2006/0161063 A1
(43) Pub. Date: Jul. 20, 2006

(54) System and method for real-time microcirculation diagnosis

(76) Inventor: Yio-Wha Shau, Taipei (TW)

Correspondence Address: LIN & ASSOCIATES INTELLECTUAL PROPERTY, P.O. BOX 2339, SARATOGA, CA, 95070-0339, US

(21) Appl. No.: 11/038,919

(22) Filed: Jan. 19, 2005

(51) Int. Cl. A61B 5/02; G06K 9/00

(52) U.S. Cl. 600/504; 600/473; 382/128

(57) ABSTRACT

Disclosed is a system and method for real-time microcirculation diagnosis. The system comprises a capillary photographic device, an image capturing device, and a data processing unit. By way of the system, informations about movement behavior of a single red blood cell and the situation of a single capillary are obtained. The method of the present invention comprises the steps of analyzing gray scale of capillary imagine pictures, and producing analytical data and diagram for monitoring movement behavior of a single red blood cell and the situation of a single capillary. The present invention improves the disadvantages of the conventional systems or methods, which only provide an average velocity of red blood cells in a microcirculation system. The system of the present invention does not need a hardware or device for Doppler analysis, and thus decreasing a hardware cost. The present invention can also provide more data and more valuable physiological informations.

### BACKGROUND OF THE INVENTION

1. Field of the Invention

The present invention relates to a system for real-time microcirculation diagnosis, particularly, to a system being capable of monitoring the movement behaviors of a single red blood cell along a single capillary. Moreover, the system can be further equipped with other devices for treating physiological conditions, such as drug delivering devices or temperature controlling devices, to quantify the capillary function for clinical reference and the physiological role played by each individual capillary.

2. The Related Art

Microcirculation system consisted by many capillaries in skin surface and organs plays an important physiological role. Capillaries bring nutrients and energies to body tissues and take the metabolic products away. Distribution of capillaries in soft tissues may vary with their functions and the conditions of organism body. Different capillaries may play different physiological roles even in the same tissue. For example, some capillaries are responsible for nutrient supplement, and some capillaries are involved with temperature control of body surface. It is helpful to understand capillary situations of an individual by monitoring the blood flow velocity in microcirculation system. For instance, capillary sclerosis may lead to a slower flow velocity of blood cells, and lesions of blood cells may cause the change in blood flow velocity. To clinical trials for some drugs, monitoring of blood flow velocity in microcirculation system also provides important physiological informations.

Currently, the most common apparatus for monitoring the flow velocity of microcirculation system is laser Doppler velocimeter. The commercial laser Doppler velocimeters, such as from PERIMED or MOOR, sent an incident low-power laser light directed form an optic fiber toward a tissue of interest, such as skin, and collects the scattered radiation to show a regional microcirculation flow of a tissue of interest. According to the Doppler principle, the incident laser beam is scattered by the movement of red blood cells (RBCs) in capillaries, the frequency of the scattered beam is Doppler shifted, and the shifted frequency is a function of the relative velocity of red blood cells. Thus, the velocity of red blood cells showing the regional blood flow can be obtained. The laser Doppler velocimeter only detects an average blood flow velocity of a tissue region. However, differences in capillary diameter and surrounding tissue result in the differences in blood flow among the capillaries, and the averaged blood flow velocity cannot reveal the physiological role of capillary individually. Thus, the conventional laser Doppler velocimeter only provides limited physiological information.

Infrared video capillaroscopy from KK Technology is another type for microcirculation monitoring. It uses an infrared photographer to record a capillary imagine video around a skin surface region and displays a dynamic movement of red blood cells in capillaries on a monitor. For further analysis, some software tools are available to analyze the capillary area, density and diameter from the infrared images. To determine blood flow velocity, image correlation technique and infrared Doppler effect are usually used to analyze the infrared images. The image correlation technique provides an average moving velocity of red blood cells by reckoning parameters of moving shift and transit time obtained from a sequence of images showing the highest correlation. But, the technique still cannot distinguish the physiological role played by each capillary. Furthermore, a tiny body movement will bring noises into detected result in the two aforementioned analysis methods, and it will influence the detection accuracy.

### SUMMARY OF THE INVENTION

The primary objective of the present invention is to provide a system for real-time microcirculation diagnosis, which can accurately evaluate functions and situations of each capillary in microcirculation system and estimate a red blood cell to be lesion or not. The system comprises a capillary photographic device, which can take capillary dynamic images; an image capturing device, which is connected to the capillary photographic device and transforms the dynamic images into a sequence of pictures; and a data processing unit comprising a data recorder, an image analyzer and a data output unit; wherein, the capillary photographic device takes the capillary dynamic images and transmits to the image capturing device, the image capturing device transforms the dynamic images into a sequence of pictures and transmits the pictures to the data recorder, the data recorder records the pictures, the image analyzer analyzes capillary gray scale gradients in the pictures to produce data or analytical diagrams for monitoring movement behaviors of a single blood red cell and situations of a single capillary, and the data output unit outputs the data and analytical diagrams.

There is no particular limitation to the capillary photographic device, as long as it can take the dynamic images of the capillaries. It may be a commercial infrared video capillaroscopy, for example, a video capillaroscopy (VCS) from KK Technology.

In order to analyze the capillary gray scale gradient in pictures, first, select a capillary region of interest from the pictures, rescale the selected region and correct with a ratio scale, mark an analyzed area with a direction being parallel to the capillary longitudinal orientation, and analyze the gray scale gradient in pictures. According to the aforementioned method, analyze gray scale gradient in the sequential pictures in a period of time. Plot a diagram of red blood cell position versus time, and obtain a “RBC (red blood cell) real-time motion mode diagram” representing a moving trace of red blood cell. RBC shifting velocity and RBC shifting acceleration are also obtained from the diagram. The diagram, shifting velocity and shifting acceleration can quantitatively reflect a moving trace of each red blood cell passing through capillary curvature, and also the changes with time. A change of RBC shifting velocity shows a resistant property of microcirculation system in the area of interest. For example, RBC shifting velocity of red blood cells decrease when they pass through a particular position, it shows that there exists a higher resistance around the position, and suggest that a capillary sclerosis or calcification may occur around the position. Also, the RBC real-time motion mode diagrams of some red blood cells with lesion are different from the normal cells. The information of numbers of red blood cells with lesion can be used to understand the healthy state of a person. Clinically, in a period of a trial or treatment, changes of RBC real-time motion mode diagrams in a predetermined capillary region reveal the effect of the treatment. The system of the present invention do not need a Doppler hardware device, therefore, the cost of hardware is effectively decreased. Moreover, the system can focus on a single capillary or a single red blood cell to obtain more important physiological parameters more accurately.

In practice, even a slight body movement may affect the result of capillary blood flow analysis, therefore, a position correlation analysis is performed between two sequential pictures to correct the noise caused by tiny body movement.

The system of the present invention can be equipped with other physiological monitoring devices to investigate a correlation among the physiological parameters. For example, arterial blood pressure measuring device, pulse oximeter, temperature controller or infrared body temperature sensor can be used with the system of the present invention together to monitor the microcirculation of hands or feet. All the physiological parameters obtained from those devices can be transmitted to the data processing unit to be analyzed. Studying the physiological relationship among those parameters is advantageous to investigate the microcirculation system.

For easy operation, the data processing unit may be a computer.

Another objective of the present invention is to provide a method for real-time microcirculation diagnosis, which comprises the steps of (a) providing a system for real-time microcirculation diagnosis, which comprises a capillary photographic device, an image capturing device, and a data processing unit comprising a data recorder, an image analyzer and a data output unit; (b) using the capillary photographic device to take capillary dynamic images of a predetermined region; (c) transmitting the images to the image capturing device; (d) transforming the images into a sequence of image pictures with the image capturing device, and transmitting the pictures to the data recorder to record the pictures; (e) selecting a capillary in the pictures and analyzing a gray scale gradient of the selected capillary to produce data or analytical diagrams, which can monitor movement behaviors of a single blood red cell and situations of a single capillary; and (f) outputting the data and analytical diagrams with the output unit.

For overcoming a noise derived from a body movement during a diagnosis period, step (c) can further comprises a step of performing a correlation analysis between two sequential pictures, and thus correcting the movement noise. In order to analyze the gray scale gradient in pictures, first, select a capillary region of interest from the pictures, rescale the selected region and correct with a ratio scale, mark an analyzed area with a direction being parallel to the capillary longitudinal orientation, and analyze the gray scale gradient in pictures. According to the aforementioned method, analyze the sequential gray scale gradient in pictures in a period of time. Plot a diagram of red blood cell position versus time, and obtain a RBC real-time motion mode diagram representing a movement trace of red blood cell. RBC shifting velocity and RBC shifting acceleration of red blood cells are also obtained from the diagram.

The present invention is further explained in the following embodiment illustration and examples. It is realized that these are not to be construed as limiting the scope of the invention but as merely providing illustrations of some of the presently preferred embodiments of this invention. The person skilled in the art may make various modifications and changes without departing from the scope and spirit of the present invention.

### BRIEF DESCRIPTION OF THE DRAWINGS

FIG. 1 depicts an embodiment of a system for real-time microcirculation diagnosisin accordance with the present invention;

FIG. 2 illustrates an analysis of the image analyzer in accordance with the present invention, and obtained data or analytical diagrams form the analysis;

FIG. 3 is another embodiment of a system for real-time microcirculation diagnosis in accordance with the present invention;

FIG. 4 is still another embodiment of a system for real-time microcirculation diagnosis in accordance with the present invention;

FIG. 5 is an example showing a diagnosed result using the system of the present invention with an arterial blood pressure measuring device and a pulse oximeter;

FIG. 6 is an example showing a diagnosed result using the system of the present invention with a temperature controller and an infrared body temperature sensor; and

FIG. 7 shows a flow chart illustrating a method for real-time microcirculation diagnosis in accordance with the present invention.

### DETAILED DESCRIPTION OF THE PREFERRED EMBODIMENT

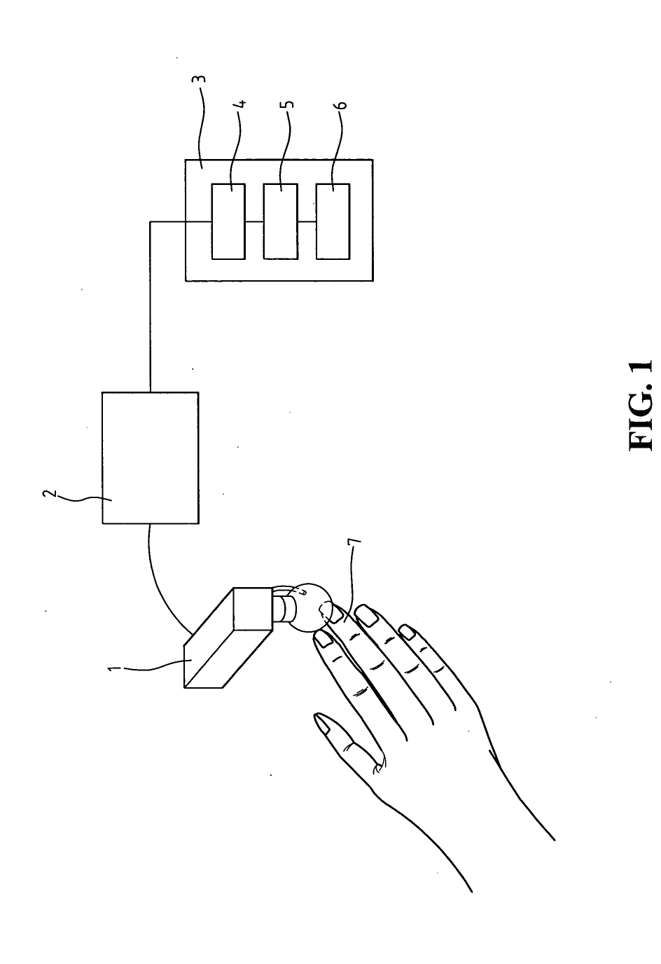

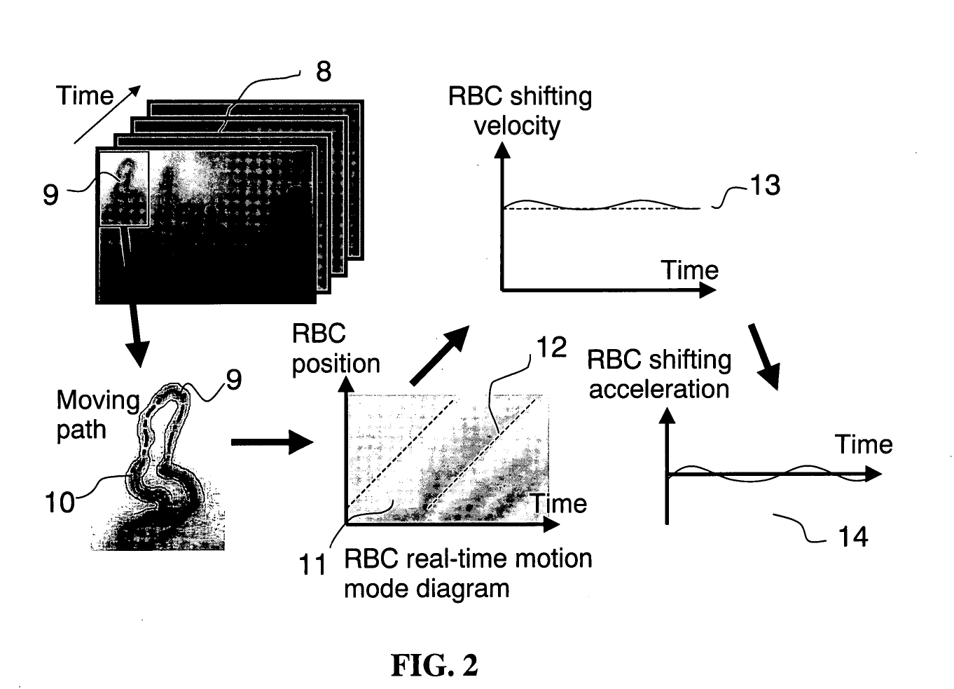

FIG. 1 depicts an embodiment of the system of the present invention, the system comprises an commercial infrared capillary photographer 1, an image capturing device 2 and a data processing unit 3 comprising a data recorder 4, an image analyzer 5 and a data output device 6. As an example, the system is applied to diagnose a microcirculatory region 7 in a fingernail. Images taken by the capillary photographer 1 are transmitted to the image capturing device 2, image capturing device 2 transforms the imagines into a sequence of imagine pictures and transmit the pictures to the data processing unit 3, the data recorder 4 records those pictures. The data analyzer 5 analyzes gray scale gradients of the pictures and produces analytical data and diagrams as shown in FIG. 2. Select a capillary 9 in the first picture of the sequence of images pictures, correct the selected region with a ration scale, mark a analyzed region and position along a direction being parallel to the longitudinal orientation of capillary (the longitudinal orientation after straitening up the capillary), and analyze the gray scale gradient of the picture. Sequential pictures taken at different time point are analyzed in the same way. Then, plot a diagram of gray scale position versus time according to a conventional gray-scale interpolation technique. Since an object with the same gray scale gradient in different picture can be considered as the same object, the moving position of the same gray scale gradient in different time point shows a moving path of a single red blood cell. The diagram is termed as “RBC real-time motion mode diagram” (diagram 11). Dashed line 12 in diagram 11 represents a moving trace of the monitored red blood cell, and the slope of dashed line 12 represents the velocity of the red blood cell. The steeper slope means the blood cell velocity is faster, and the flatter slope means the blood cell velocity is slower. A moving path 10 of the blood cell in capillary 9 can be determined in diagram 11. Analytical diagram for shifting velocity (diagram 13) and shifting acceleration (diagram 14) of red blood cells are also derived from the diagram 11. These analytical diagram can be output from a data output device 6.

To remove noises derived from a tiny body movement, a correlation analysis is performed between the two sequential pictures. The image pictures are first changed to inverse images, and image binaryzation is carried out according to a threshold value of the gray scale in pictures. Position correlation of two white dots (representing blood cells in capillaries) between two sequential pictures is calculated. The position shifting is corrected by adjusting the picture or rotating the shifting angle according to the position correlation analysis. Thus it is achievable to correct the noise caused by body movements.

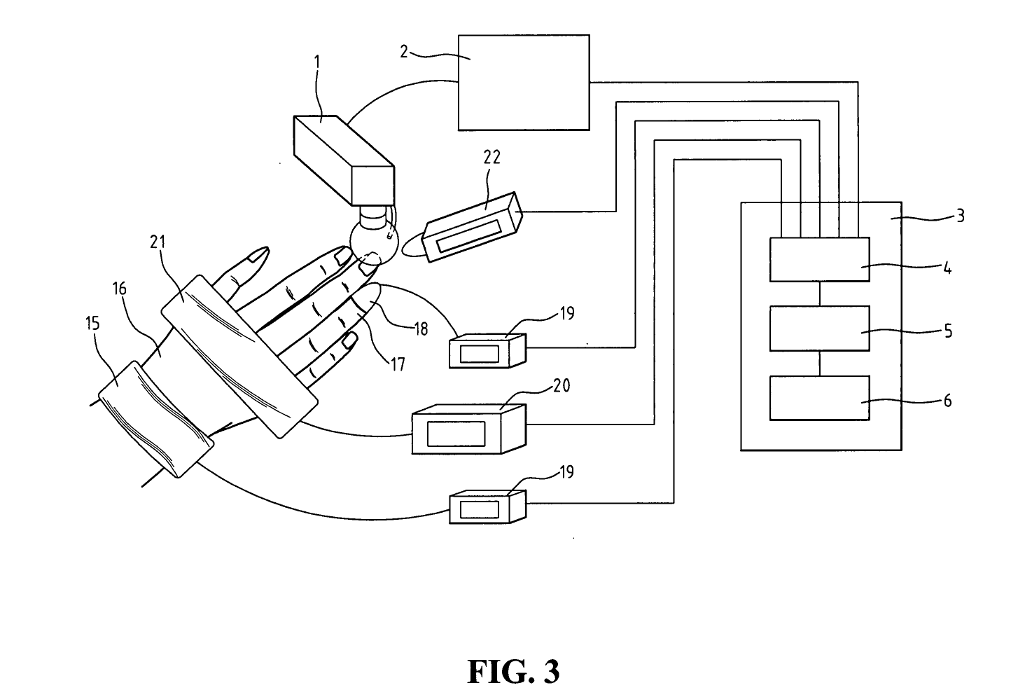

Another embodiment of the present invention is shown in FIG. 3, which is an exemplary system shown as FIG. 1 equipped with other physiological monitoring devices. As an example to diagnose a fingernail microcirculatory region, a system shown as FIG. 1 is set up. Furthermore, an arterial blood pressure measuring device 15 is placed on the radius artery (upstream of the capillary 16) to measure the blood pressure signals, and an oximeter 18 is placed on another finger capillary 17 to obtain the pulse wave signals monitoring percent of oxyhemoglobin. The two signals are transmitted to an A/D converter 19 to become digital signals, and then passed to the data processing unit 3 for further analysis. Also, a temperature control device 20 connecting to a thermo-controlled ice bag 21 is placed on the upstream portion of the capillary, such as the arm or palm. An infrared body temperature sensor 22 can also be equipped with the system to detect the body surface temperature around the region of finger's capillaries, and the measured data are also passed to the data processing unit 3. After correcting recording time differences of each devices, all the synchronous taken signals including capillary images, artery pulse waves, controlled temperature by temperature controller, and body surface temperature around the region of finger's capillaries are all fed to the data processing unit 3 at the same time to perform image and data analysis. For easy operation, the data processing unit 3 may be a computer.

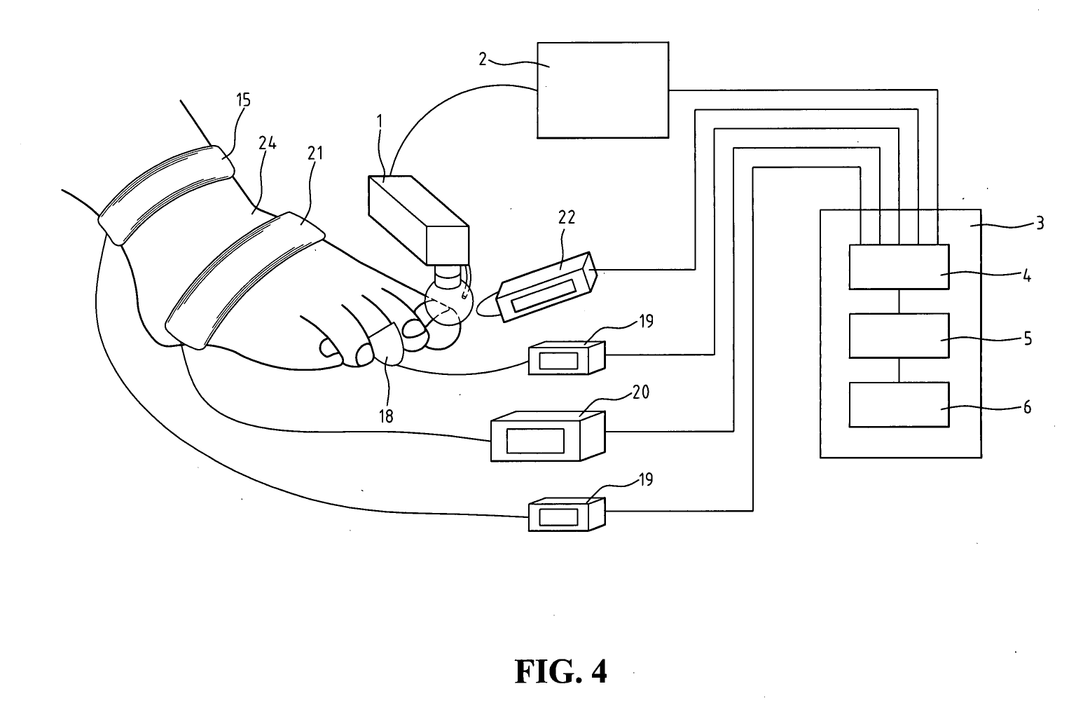

Another embodiment for diagnosing a foot microcirculation is shown as FIG. 4. An infrared capillary photographer 1 takes imagines of capillary 23 in a toe. The imagines is transmitted to an image capturing device 2 to capture a sequence of imagine pictures. Also, an arterial blood pressure measuring device 15 is placed on the dorsalis pedis artery (upstream of the capillary 16) to measure the blood pressure signals, and an oximeter 18 is placed on another toe to obtain the pulse wave signals monitoring percent of oxyhemoglobin. The two synchronous signals are transmitted to an A/D converter 19 to become digital signals, and then passed to the data processing unit 3 for further analysis. Moreover, a thermo-controlled ice bag 21 is placed on the dorsalis pedis to control temperature. An infrared body temperature sensor 22 is also equipped with the system to detect the body surface temperature around the region of finger's capillaries, and the measured data are also passed to the data processing unit 3.

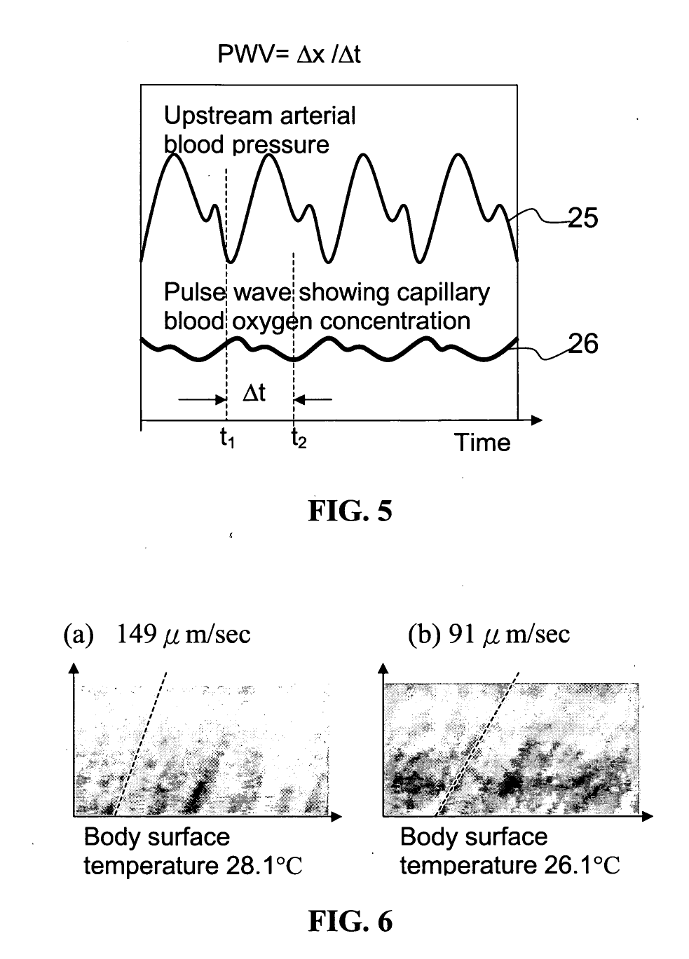

An exemplary measuring result showing an arterial blood pressure waveform and a pulse wave of blood oxygen concentration with the microcirculation diagnosis system equipped with an arterial blood pressure measuring device and an oximeter is shown in FIG. 5. Red blood cells in capillaries will pulse slightly since a driving force from heart pulse pressure. An upstream arterial blood pressure waveform 25 is obtained with an arterial blood pressure measuring device, a pulse wave 26 showing capillary blood oxygen concentration is obtained with an oximeter, and shifting distances of red blood cells are obtained with the microcirculation diagnosis system. Then, a time point corresponding to a wave valley in upstream arterial blood pressure waveform 25 is defined as t₁, a time point corresponding to a next wave valley in a pulse wave 26 is defined as t₂, and a time delay between t, and t₂ is defined as Δt. From the detected result with microcirculation diagnosis system, Δx representing a shifting distance of a red blood cell in a time interval Δt can be obtained. A parameter of PWV (pulse wave velocity) is also obtained by the following formula:
PWV=Δx/Δt
PWV is a parameter relating to mechanical properties of blood vessel and a state of microcirculation system.

An exemplary measuring result showing using the microcirculation diagnosis system equipped with an infrared body temperature sensor is shown in FIG. 6. With comparison of RBC real-time motion mode diagrams between two different controlled body surface temperatures, slops of gray scale in pictures show that the shifting velocity of red blood cell in capillary is decelerated with a decreased body temperature. It suggests that soft tissues will contract with a decreased body surface temperature, and thus changing the property of capillary. There are great interactions between the blood flow in capillaries and peripheral tissue environments. When the body surface temperature is changed, traces and shifting velocity of red blood cells flowing toward body surface or interior tissues are also changed. On the other side, after removing a temperature treatment, metabolic reactions in soft tissues will bring the blood flow in capillaries back to a normal state, the time needed to be back to a normal state and a curve showing blood flow velocity back to a normal state are meaningful in physiology. Also, a decreased body temperature will lead to a changing of blood flow properties and influence artery pulse wave velocity. Studies of temperature effect on a regional (artery) blood flow can reflect transporting mechanism and changing of (artery) circulation system, thus obtaining quantified parameters representing functions of a regional (artery) circulation system.

All the parameters obtained from the above-mentioned assays, such as PWV, blood flowing velocity in microcirculation system, and the body surface temperature, can be a clinical index or useful reference for classification of microcirculatory diseases. More physiological parameters are advantageous to precisely realize a patient's microcirculatory situation. For example, to diagnose a microcirculation system of diabetic foot, the aforementioned detection embodiment provides a method more conveniently and precisely. It is also a better way to monitor an effect of clinical therapy or medication usage.

Except for geometrical characteristics of capillaries, embodiments shown in FIGS. 3 and 4 also provide other important physiological data, such as: (1) RBC real-time motion mode diagram, RBC shifting velocity along capillary, and RBC shifting velocity along capillary; (2) changing of RBC shifting velocity in each regional capillary and the related analysis data; (3) PWV of blood flow from upstream artery to downstream capillary; (4) real-time detected results showing the effect of temperature treatment in blood upstream on downstream RBC velocity in capillary; (5) real-time detected results showing effect of temperature treatment in blood upstream on PWV of downstream capillary; (6) recovery time being back to a normal state of RBC velocity or PWV after removing the temperature treatment in blood upstream.

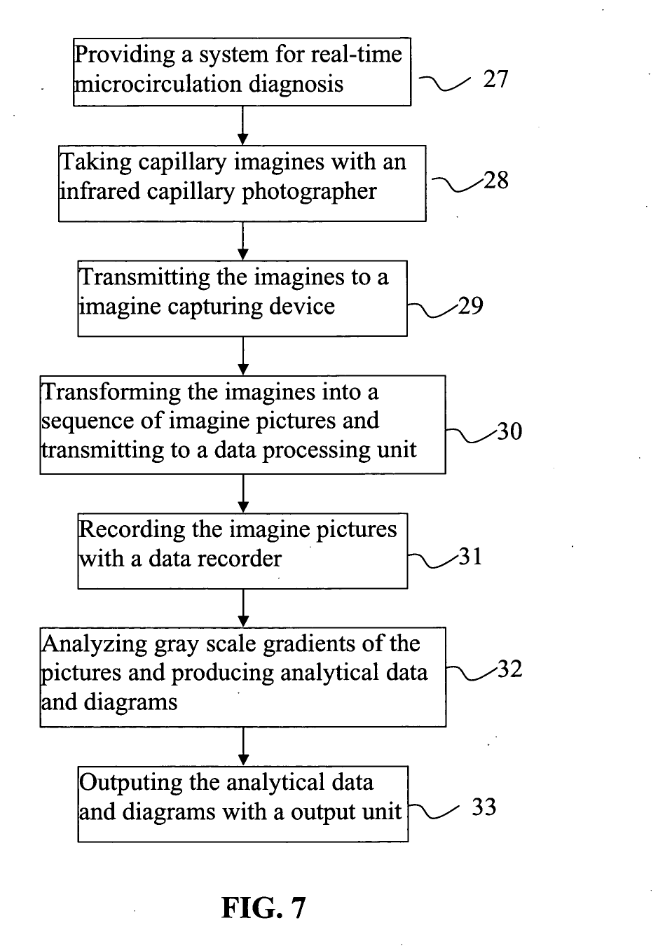

Particularly, the method for real-time microcirculation diagnosis provided by the present invention is shown as FIG. 7. First, an aforementioned system for real-time microcirculation diagnosis is provided (step 27); images of the capillary of interest are taken by an infrared capillary photographer (step 28); the taken images are transmitted to a imagine capturing device (step 29); the imagines are captured as a sequence of imagine pictures, which are transmitted to a data processing unit (step 30); the imagine pictures are recorded as a pictures by a data recorder (step 31); the gray scale gradient of the pictures are analyzed, and analytical data and diagrams are produced (step 32); and the analytical data and diagrams are output with a output unit(step 33).

In the analysis step 32, a selected region in picture is rescaled and corrected with a ration scale, an analyzed region and position along a direction being parallel to the longitudinal orientation of capillary is marked, and the geometrical parameters of capillaries (such as vessel diameter, length and curvature) are recorded to be references for clinical correlation comparison. Then, gray scale gradients of the sequential pictures at different time point are analyzed in a same way. Imagine shifting caused by body movement is also corrected according to a result of correlation analysis between two sequential pictures. A diagram of gray scale position versus time is plotted according to an interpolation technique, and the diagram termed “RBC real-time motion mode diagram” representing a moving trace of red blood cell is obtained. A slope obtained according to a gray scale threshold along the time axis of the real-time motion mode diagram represents a RBC shifting velocity along capillary. A slope of the shifting velocity with the time represents a RBC shifting acceleration along capillary. All the analytical data and diagrams can be either output from an output unit directly, or be output after calculating or performing a statistic analysis with other physiological parameters.

The RBC real-time motion mode diagram provided by the present invention can monitor a moving trace of each red blood cell passing through a capillary curve and changes of the moving trace with time. Moreover, there exists large variations on shifting velocity of each red blood cell since the differences exists in capillary diameter and environmental pressure. The present invention not only can obtain an average RBC velocity, but also can focus on each red blood cell or each capillary to study microcirculation in more details, thus providing more important physiological data about microcirculation system. From a regional blood flow velocity and the data of capillary diameter, those data can be used to realize the difference and characteristics among the capillaries. According to the geometrical characteristics and distribution relationship in a region of capillaries, the system can identify a specific capillary easily. It can be apply to monitor a treatment effect clinically, for example, changes of the RBC real-time motion mode diagram and shifting velocity can suggest the effect for a treatment.

From the descriptions above, comparing to the conventional system or method, the present invention provides a system and method diagnosing a microcirculation system with more precise analytical data and diagrams for realizing moving trace of a single red blood cell and situation of a single capillary but not only provide an average velocity of red blood cells. The present invention can provide more data and more valuable physiological informations. Furthermore, the present invention can remove noises derived from body movement by correction, and thus resulting in a result more accurately. Also, the system of the present invention does not need a hardware or device for Doppler analysis, and thus decreasing a hardware cost.

What is claimed is:

1. A system for real-time microcirculation diagnosis, comprising:
a capillary photographic device, which takes capillary dynamic images;
an image capturing device, which is connected to the capillary photographic device and transforms the dynamic images into a sequence of pictures; and
a data processing unit comprising a data recorder, an image analyzer and a data output unit;
wherein, the capillary photographic device takes the capillary dynamic images and transmits to the image capturing device, the image capturing device transforms the dynamic images into a sequence of pictures and transmits the pictures to the data recorder, the data recorder records the pictures, the image analyzer analyzes capillary gray scale gradients in the pictures to produce data or analytical diagrams for monitoring movement behaviors of a single blood red cell and situations of a single capillary, and the data output unit outputs the data and analytical diagrams.

2. The system as claimed in claim 1, wherein the capillary photographic device is an infrared video capillaroscopy.

3. The system as claimed in claim 1, wherein the data processing unit is a computer.

4. The system as claimed in claim 1, wherein the data or analytical diagrams comprise a RBC real-time motion mode diagram.

5. The system as claimed in claim 1, wherein the data or analytical diagrams comprise a data of RBC shifting velocity.

6. The system as claimed in claim 1, wherein the data or analytical diagrams comprise a data of RBC shifting acceleration. 7.

7. A method for real-time microcirculation diagnosis, comprising the steps of:
(a) providing a system for real-time microcirculation diagnosis comprising a capillary photographic device, an image capturing device, and a data processing unit comprising a data recorder, an image analyzer and a data output unit;
(b) using the capillary photographic device to take capillary dynamic images of a predetermined region;
(c) transmitting the images to the image capturing device;
(d) transforming the images into a sequence of image pictures with the image capturing device, and transmitting the pictures to the data recorder to record the pictures;
(e) selecting a capillary in the pictures and analyzing a gray scale gradient of the selected capillary to produce data or analytical diagrams; and
(f) outputting the data and analytical diagrams with the output unit.

8. The method as claimed in claim 7, wherein the capillary photographic device is an infrared video capillaroscopy.

9. The method as claimed in claim 7, wherein the data processing unit is a computer.

10. The method as claimed in claim 7, wherein the data or analytical diagrams comprise a RBC real-time motion mode diagram.

11. The method as claimed in claim 7, wherein the data or analytical diagrams comprise a data of RBC shifting velocity.

12. The method as claimed in claim 7, wherein the data or analytical diagrams comprise a data of RBC shifting acceleration.

13. The method as claimed in claim 7, wherein step (e) comprises the steps of:
(i) selecting a capillary region from the pictures;
(ii) correcting the selected capillary pictures with a ration scale;
(iii) marking an analyzed capillary area along a direction being parallel to the longitudinal orientation of capillary;
(iv) analyzing gray scale gradients of the sequential pictures at different time point;
(v) plotting a diagram of gray scale gradient position versus time; and
(vi) obtaining a RBC real-time motion mode diagram.

14. The method as claimed in claim 13, wherein step (e) further comprises obtaining a data of RBC shifting velocity from the RBC real-time motion mode diagram.

15. The method as claimed in claim 14, wherein step (e) comprises obtaining a diagram of RBC shifting velocity versus time.

16. The method as claimed in claim 13, wherein step (e) further comprises obtaining a data of RBC shifting acceleration from the RBC real-time motion mode diagram.

17. The method as claimed in claim 16, wherein step (e) comprises obtaining a diagram of RBC shifting acceleration versus time.

18. The method as claimed in claim 13, which further comprises correcting a imagine picture shifting derived from a tiny body movement after step (iii).

19. The method as claimed in claim 18, which comprises the steps of:
(1) changing the image pictures to inverse video;
(2) performing an image binaryzation according to a predetermined threshold value of the gray scale in the imagine pictures;
(3) calculating a position correlation of two white dots between two sequential pictures; and
(4) correcting the image picture shifting by adjusting a picture position according to a analyzed result of step (3).

20. The method as claimed in claim 19, wherein step (4) comprises shifting the picture or rotating a shifting angle.
